(19) United States
(12) Patent Application Publication
(10) Pub. No.: US 2024/0423484 A1
(43) Pub. Date: Dec. 26, 2024

(54) SYSTEMS AND METHODS FOR MEASURING CARDIAC AND RESPIRATORY SIGNALS

(71) Applicant: Apple Inc., Cupertino, CA (US)

(72) Inventors: Izzet B. YILDIZ, Sunnyvale, CA (US); Grant H. MULLIKEN, San Jose, CA (US); Jonathan HADIDA, Redwood City, CA (US); Dimitri E. DIAKOPOULOS, San Jose, CA (US); Brian PASLEY, Berkeley, CA (US)

(21) Appl. No.: 18/693,924

(22) PCT Filed: Sep. 13, 2022
(86) PCT No.: PCT/US2022/043389

§ 371 Date: Mar. 20, 2024

Related U.S. Application Data
(60) Provisional application No. 63/248,356, filed on Sep. 24, 2021.

(51) Int. Cl. A61B 5/0205; A61B 5/00; A61B 5/024; A61B 5/11; A61B 5/113; G06T 7/20

(52) U.S. Cl. CPC A61B 5/0205; A61B 5/02433; A61B 5/1114; A61B 5/1128; A61B 5/113; A61B 5/6814; A61B 5/725; A61B 5/7445; G06T 7/20; A61B 2562/0219; A61B 2562/185; G06T 2207/10048; G06T 2207/30196

(57) ABSTRACT

A wearable electronic device may be provided that includes a first image sensor oriented to capture luminance of an area of skin around a user's eye when wearing the wearable electronic device and a second image sensor oriented to capture luminance of an area of skin around the user's nose when wearing the wearable electronic device. The wearable electronic device further includes a processor configured to determine a pulse signal based on changes in luminance captured over time using at least one of the first image sensor or the second image sensor.

### CROSS-REFERENCE TO RELATED APPLICATION

This application claims the benefit of U.S. Provisional Application No. 63/248,356, entitled “SYSTEMS AND METHODS FOR MEASURING CARDIAC AND RESPIRATORY SIGNALS,” filed Sep. 24, 2021, the entirety of which is incorporated herein by reference.

### TECHNICAL FIELD

The present description relates generally to electronic devices including, for example, systems and methods for measuring cardiac and respiratory signals using electronic devices.

### BACKGROUND

Heart and respiratory signals may be estimated using electronic devices specially designed for this purpose. These specialized electronic devices typically require close contact with the body and skin and therefore may be irritating or distracting for users.

### BRIEF DESCRIPTION OF THE DRAWINGS

Certain features of the subject technology are set forth in the appended claims. However, for purpose of explanation, several embodiments of the subject technology are set forth in the following figures.

FIG. 1 is a block diagram depicting components of a head-mountable device according to aspects of the subject technology.

FIG. 2 is a diagram illustrating regions of interest captured by image sensors of a head-mountable device being worn by user according to aspects of the subject technology.

FIG. 3 is a flowchart illustrating an example processing for measuring a heart rate or respiratory rate according to aspects of the subject technology.

FIG. 4 depicts a luminance signal and a ground-truth respiration signal according to aspects of the subject technology.

FIG. 5 depicts examples of respiratory analysis of a signal according to aspects of the subject technology.

FIG. 6 illustrates a block diagram of a head-mountable device, in accordance with some embodiments of the present disclosure.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be clear and apparent to those skilled in the art that the subject technology is not limited to the specific details set forth herein and may be practiced without these specific details. In some instances, well-known structures and components are shown in block diagram form in order to avoid obscuring the concepts of the subject technology.

Head-mountable devices such as head-mountable displays typically include a combination of cameras oriented to capture different regions of interest with respect to a wearing user. For example, images captured by the cameras may be used for localization and mapping of the head-mountable device in its environment, tracking hand/body pose and movements, tracking jaw and mouth for user representation, tracking eye movements, etc. The cameras may be red green blue (RGB) cameras, infrared cameras, or a combination of these two types of cameras. The subject technology proposes to use these existing cameras in head-mountable devices in place of specialized sensors to measure heart and respiratory signals.

According to aspects of the subject technology, the cameras are used to capture luminance values of different areas of a user wearing the head-mountable device. For example, the cameras may capture the luminance of an area of skin around the user's eyes or around the user's nose. These luminance values captured over time may be used to determine a pulse signal for the wearing user. Similarly, luminance values captured over time of the user's chest may be used to determine a respiratory signal for the wearing user.

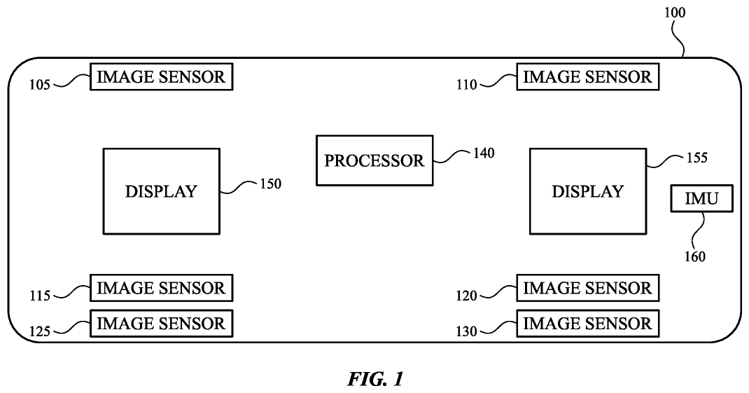

FIG. 1 is a block diagram depicting components of a head-mountable device according to aspects of the subject technology. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

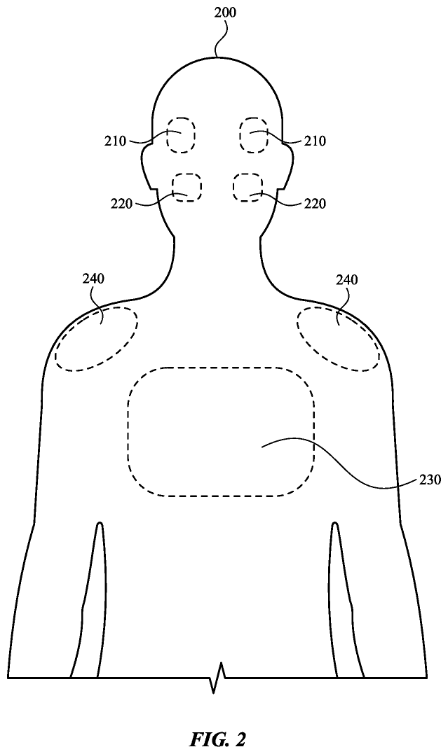

As depicted in FIG. 1, head-mountable device 100 includes image sensors 105, 110, 115, 120, 125, and 130 (image sensors 105-130), processor 140, display units 150 and 155, and inertial measurement unit (IMU) 160. Image sensors 105-130 may be individually oriented to capture different regions of interest on a wearing user's body. For example, image sensors 105 and 110 may be oriented to capture areas of skin around a wearing user's eyes as represented by regions of interest 210 of user 200 depicted in FIG. 2. Image sensors 115 and 120 may be oriented to capture areas of skin around the user nose such as the upper cheeks as represented by regions of interest 220 depicted in FIG. 2. Image sensors 125 and 130 may be oriented to capture regions of interest on the upper body of a wearing user such as the chest area and shoulders as represented by regions of interest 230 and 240 depicted in FIG. 2. Image sensors 105-130 may be infrared image sensors and may have associated infrared illuminators to illuminate the respective regions of interest with infrared light.

Processor 140 may include suitable logic, circuitry, and/or code that enable processing data and/or controlling operations of head-mountable device 100. In this regard, processor 140 may be enabled to provide control signals to various other components of head-mountable device 100. Processor 140 may also control transfers of data between various portions of head-mountable device 100. Additionally, processor 140 may enable implementation of an operating system or otherwise execute code to manage operations of head-mountable device 100. Processor 140 or one or more portions thereof, may be implemented in software (e.g., instructions, subroutines, code), may be implemented in hardware (e.g., an Application Specific Integrated Circuit (ASIC), a Field Programmable Gate Array (FPGA), a Programmable Logic Device (PLD), a controller, a state machine, gated logic, discrete hardware components, or any other suitable devices) and/or a combination of both.

Display units 150 and 155 are configured to display visual information to a wearing user. Display units 150 and 155 can provide visual (e.g., image or video) output. Display units 150 and 155 can be or include an opaque, transparent, and/or translucent display. Display units 150 and 155 may have a transparent or translucent medium through which light representative of images is directed to a user's eyes. Display units 150 and 155 may utilize digital light projection, OLEDs, LEDs, uLEDs, liquid crystal on silicon, laser scanning light source, or any combination of these technologies. The medium may be an optical waveguide, a hologram medium, an optical combiner, an optical reflector, or any combination thereof. In one embodiment, the transparent or translucent display may be configured to become opaque selectively. Projection-based systems may employ retinal projection technology that projects graphical images onto a person's retina. Projection systems also may be configured to project virtual objects into the physical environment, for example, as a hologram or on a physical surface. The head-mountable device 100 can include an optical subassembly configured to help optically adjust and correctly project the image-based content being displayed by display units 150 and 155 for close up viewing. The optical subassembly can include one or more lenses, mirrors, or other optical devices.

IMU 160 is a sensor unit that may be configured to measure and report specific force, angular rate, and/or orientation of head-mountable device 100 while being worn by a user. IMU 160 may include a combination of accelerometers, gyroscopes, and/or magnetometers.

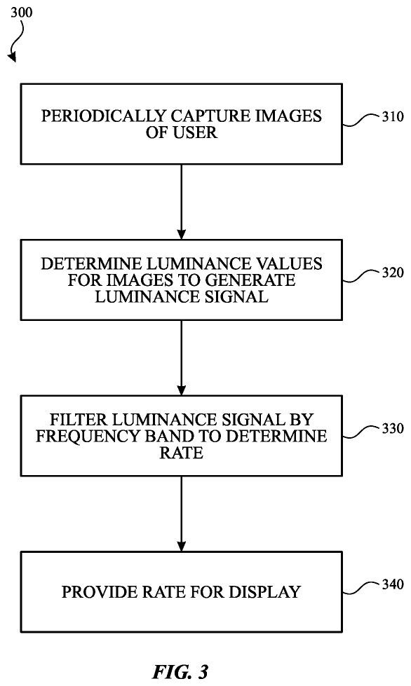

FIG. 3 is a flowchart illustrating an example processing for measuring a heart rate or respiratory rate according to aspects of the subject technology. For explanatory purposes, the blocks of process 300 are described herein as occurring in serial, or linearly. However, multiple blocks of process 300 may occur in parallel. In addition, the blocks of process 300 need not be performed in the order shown and/or one or more blocks of process 300 need not be performed and/or can be replaced by other operations.

Process 300 includes periodically capturing images of a user with one or more of image sensors 105-130 of head-mountable device 100 while being worn by the user (block 310). For example, image sensors 105 and/or 110 may capture images of an area of skin around the user's eyes. As noted above, the image sensors may be infrared image sensors and may have an associated infrared illuminator to illuminate the region of interest (e.g., area of skin around the user's eyes). Head-mountable device 100 may include a light seal configured to block external light sources from the user's eyes while wearing head-mountable device 100 and therefore minimize interference with the image sensors capturing the region of interest around the user's eyes.

Images are captured periodically by the image sensors. The rate at which the images are captured may be limited by the capabilities of the image sensors and the processing power available to process the images. Images may be captured at a rate of 24 or 30 frames per second, for example. Lower rates, such as 10 or 5 frames per second, may be used to preserve processing power for other uses while maintaining a high enough sample rate relative to the expected heart rates and/or respiratory rates. These rates represent examples and are not intended to limit the subject technology.

The amount of light reflected by the skin changes depending on the underlying blood volume and correlates well with heart cycles. For each image captured by the image sensors, a luminance value is determined and the series of luminance values from the corresponding images is used to generate a luminance signal (block 320). The luminance value may be an average luminance value of all of the pixels capturing the region of interest, such as the area of skin around the eyes of the user, for example. The luminance signal may be recorded or stored in a memory as a pulse signal.

In order to remove noise and to focus on the rate of interest, the generated luminance signal may be filtered by a frequency band (block 330). For example, the luminance signal may be filtered by a frequency band of 1 to 2.5 Hz for cardiac signals or heart rate. This frequency band is intended to be an example and different frequency bands may be used within the scope of the subject technology. The heart rate may be determined based on the frequency of peaks in the filtered luminance signal. The filtered luminance signal or a number indicating the heart rate may then be provided for display to the user on the display units of the head-mountable device worn by the user or on another electronic device in communication with the head-mountable device (block 340). Alternatively, or in addition, the heart rate may be used in conjunction with an app such as a relaxation, meditation, or fitness app being executed on the head-mountable device or another electronic device in communication with the head-mountable device.

The examples described above reference capturing images of an area of skin around the user's eyes to determine the user's heart rate. The same process may be used to determine the user's heart rate using images of an area of skin around the user's nose. For example, image sensors 115 and 120 oriented to capture areas of skin around the user nose such as the upper cheeks as represented by regions of interest 220 depicted in FIG. 2 may be used to capture images and determine the user's heart rate based on the luminance values associated with those images.

Images captured with one image sensor may be used alone or in combination with images captured by a second image sensor to determine the user's heart rate. The two image sensors may be oriented to capture areas of skin on different sides of the user's face, such as image sensors 105 and 110 each being oriented to capture an area of skin around a different respective eye, or image sensors 115 and 120 each being oriented to capture an area of skin on a different side of the user's nose. In addition, images of the areas of skin around the user's eyes may be used in combination with images of the areas of skin around the user's nose. When multiple sets of images are used, the luminance values associated with concurrently captured images may averaged or combined in another manner to generate the luminance values to generate the luminance signal.

The foregoing examples discussed the use of luminance values from images of a user's skin to determine heart rate based on the concept that the reflected light amount changes depending on the underlying blood volume. According to other aspects of the subject technology, changes in luminance due to movement of the user may be used to determine a respiratory rate for the user. As the user breathes, subtle head movements cause changes in luminance on the user's face that may be captured using image sensors 115 and 120 oriented towards areas of skin around the user's nose. Using the process above, a luminance signal may be generated based on a series of images captured by the image sensors and the associated luminance values. This luminance signal may be recorded or stored in a memory as a respiratory signal. The luminance signal may be filtered by a frequency band such as 0.1 to 0.6 Hz to remove noise and focus on the respiratory rate. This frequency band represents one example and the subject technology may be practiced using other frequency bands. The respiratory rate may be determined based on the peaks of the filtered luminance signal. Similar to the determined heart rate, the respiratory rate may be provided for display to the user or used in conjunction with an app such as a relaxation, meditation, or fitness app being executed on the head-mountable device or another electronic device in communication with the head-mountable device.

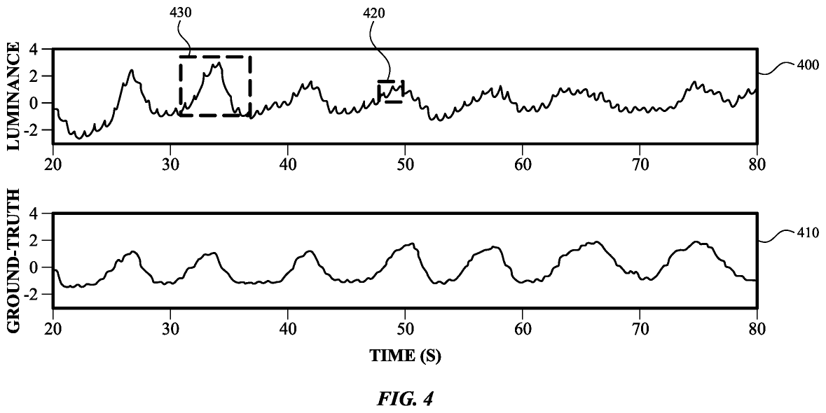

FIG. 4 depicts a luminance signal and a ground-truth respiration signal according to aspects of the subject technology. As depicted in FIG. 4, graph 400 represents a luminance signal generated from luminance values from a series of images captured of an area of skin around the user's nose. Graph 410 represents a ground-truth respiration signal obtained from a respiratory sensor such as one that measures movements and pressure changes of the chest and abdominal wall of the user using elastic bands placed around the user's torso. The small peaks highlighted by square 420 represent the heart rate of the user while the large peaks highlighted by square 430 represent the respiratory rate of the user. Comparing graph 400 with graph 410 demonstrates the respiratory rate determined based on the changes in luminance correlates well with the ground-truth respiratory rate obtained using the specialized sensor.

Motion of the user's chest may be captured using image sensors 125 and 130 and used to generate a respiratory signal. According to aspects of the subject technology, a luminance signal may be generated in manner similar to that described above using images periodically captured of the user's chest area and filtered to determine a respiratory rate. The changes in the light reflected from the user's chest may be due to changes in shadow, light direction, etc. as a result of movement of the chest while breathing.

Alternatively, images of the user's upper body captured by image sensors 125 and 130 may be processed using computer-vision algorithms to track the expansion and contraction of the upper body corresponding to breathing cycles. For example, computer-vision algorithms may process images of the user's upper body to locate body joints such as the shoulders and waist of the user. The located body joints may be used to approximate regions of interest on the upper body such as region of interest 230 in FIG. 2 for the chest and regions of interest 240 in FIG. 2 for the shoulders. Feature detection may be used to identify trackable features within the regions of interest. When multiple features are detected in a region of interest, the mean of the locations of the detected features may be used as the point within that region of interest for tracking the movement of the user's upper body. Optical flow tracking may then be used to track the relative positions of a point in the chest region of interest 230 and a point in one of the shoulder regions of interest 240 and the distances between the two points over a sequence of captured images are captured to generate an oscillatory signal reflecting the respiratory activity of the user. The rate of the captured images may be limited by the capabilities of the image sensors. For example, an image sensor capturing images at a rate of 30 frames per second could provide images and the corresponding distance values for the oscillatory signal at a rate of 30 Hz. Power and/or processing limitations may further limit this rate below the frame rate of the image sensors (e.g., 5 Hz).

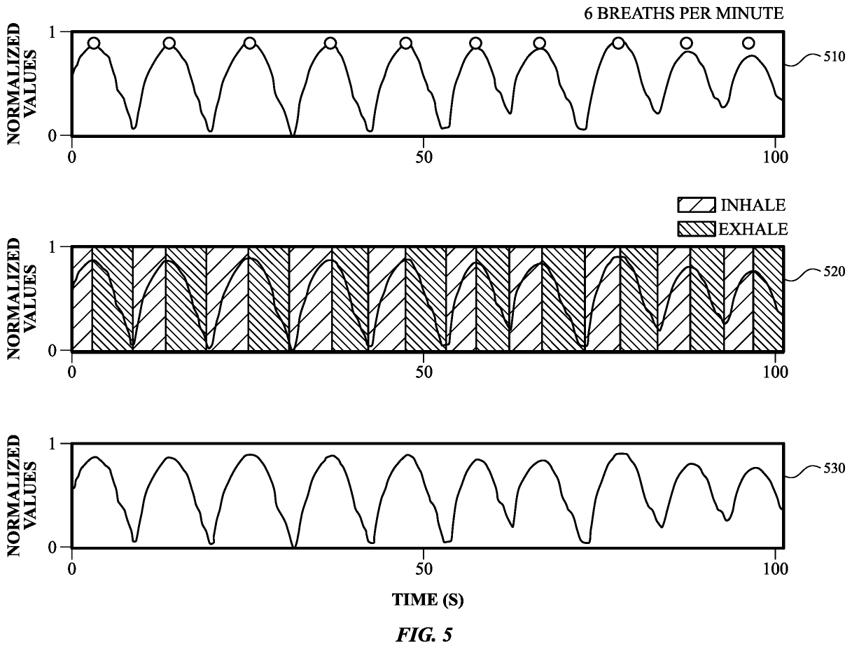

FIG. 5 depicts examples of respiratory analysis of a signal according to aspects of the subject technology. For example, graph 510 depicts a detected breath rate represented by the dots relative to a ground-truth signal captured using a mechanical chest strap worn by a user. The breath rate may be detected using data of the generated oscillatory signal described above accumulated over a period of time (e.g., one minute, five minutes, etc.). A power-spectrum analysis may be performed on the accumulated data to determine power levels of the different frequencies within the oscillatory signal. Limiting the analysis of the power spectrum to frequencies within a breathing frequency range (e.g., 4 breaths/minute to 15 breaths/minute), the frequency with the highest power level in that range may be used as the detected breath rate for the period of time (e.g., 6 breaths/minute). The detected breath rate may be detected and presented to the user at the end of a period of time or session of activity, or detected and presented periodically during the period of time or session of activity.

According to aspects of the subject technology, graph 520 depicts a segmentation of the generated oscillatory signal into periods of inhale and periods of exhale relative to the ground-truth signal. The segmentation may be performed using the derivative of the oscillatory signal, where a positive derivative indicates a rising oscillatory signal resulting from an expansion of the upper body (i.e., inhaling) and a negative derivative indicates a falling oscillatory signal resulting from a contraction of the upper body (i.e., exhaling). The segmentation into periods of inhale and periods of exhale may be determined with a shorter window of data than that described above for the breath rate detection. For example, the previous 2-3 seconds of data of the oscillatory signal may be used to determine the segmentation. The periods of inhale and the periods of exhale may be presented to the user using different visual indicators and/or different audio signals.

According to aspects of the subject technology, graph 530 depicts a full time-series approximation of the user's breathing from the generated oscillatory signal. The oscillatory signal may be noisy and may drift over time. Accordingly, the generated oscillatory signal may be normalized and smoothed to provide the full time-series approximation. For example, the mean and the variance of the generated oscillatory signal may be determined for a previous period of time (e.g., 5-10 seconds) and the oscillatory signal may be normalized by subtracting the mean from the oscillatory signal value and dividing the result by the variance.

Smoothing of the oscillatory signal may be done by averaging the signal over a previous period of time (e.g., one second). The resulting full time-series approximation may be provided for display to the user and/or provided to an application for further analysis.

According to aspects of the subject technology, image sensors 115 and 120 may be configured to capture images of the user's nostrils over time. Computer-vision algorithms may be used to track movement of the tips of the nostrils during inhalation and exhalation. This movement of the user's nostrils may be used to generate a signal for determining the respiratory rate of the user.

Respiratory signals based on the images captured of the area of skin around the user's nose may be averaged or combined in another way with the respiratory signals determined from the generated oscillatory signal described above to determine a respiratory rate of the user. In addition, measurements from IMU 160 and/or visual-inertial odometry (VIO) algorithms may be used to detect subtle head movements by the wearing user. These detected head movements may be used to generate a signal that may be combined with the respiratory signals determined from the generated oscillatory signal described above to determine the respiratory rate of the user. Furthermore, one or more microphones arranged in head-mountable device 100 may be configured to capture breathing noises from which a respiratory signal could be generated. This respiratory signal may be combined with the other respiratory signals described above to determine a respiratory rate of the user.

The head-mountable device can be worn by a user to display visual information within the field of view of the user. The head-mountable device can be used as a virtual reality system, an augmented reality system, and/or a mixed reality system. A user may observe outputs provided by the head-mountable device, such as visual information provided on a display. The display can optionally allow a user to observe an environment outside of the head-mountable device. Other outputs provided by the head-mountable device can include speaker output and/or haptic feedback. A user may further interact with the head-mountable device by providing inputs for processing by one or more components of the head-mountable device. For example, the user can provide tactile inputs, voice commands, and other inputs while the device is mounted to the user's head.

A physical environment refers to a physical world that people can interact with and/or sense without necessarily requiring the aid of an electronic device. A computer-generated reality environment relates to a partially or wholly simulated environment that people sense and/or interact with the assistance of an electronic device. Examples of computer-generated reality include, but are not limited to, mixed reality and virtual reality. Examples of mixed realities can include augmented reality and augmented virtuality. Examples of electronic devices that enable a person to sense and/or interact with various computer-generated reality environments include head-mountable devices, projection-based devices, heads-up displays (HUDs), vehicle windshields having integrated display capability, windows having integrated display capability, displays formed as lenses designed to be placed on a person's eyes (e.g., similar to contact lenses), headphones/earphones, speaker arrays, input devices (e.g., wearable or handheld controllers with or without haptic feedback), smartphones, tablets, and desktop/laptop computers. A head-mountable device can have an integrated opaque display, have a transparent or translucent display, or be configured to accept an external opaque display from another device (e.g., smartphone).

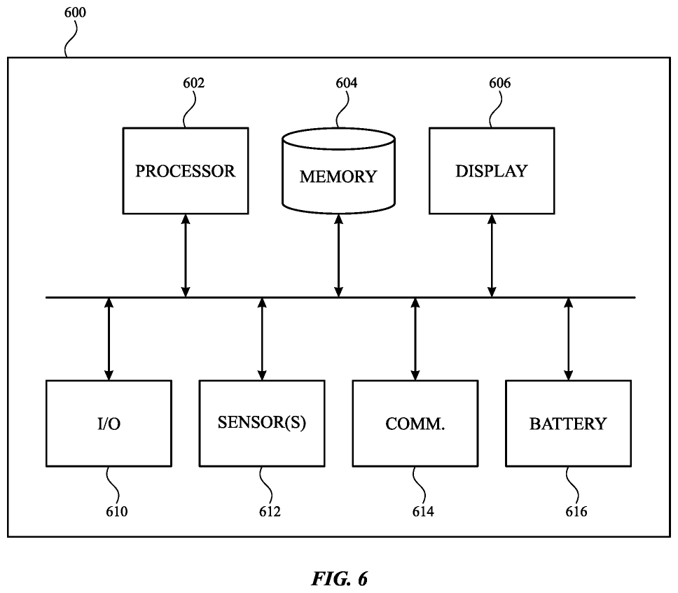

FIG. 6 is a block diagram of head-mountable device 100 according to aspects of the subject technology. It will be appreciated that components described herein can be provided on either or both of a frame and/or a securement element of the head-mountable device 100. It will be understood that additional components, different components, or fewer components than those illustrated may be utilized within the scope of the subject disclosure.

As shown in FIG. 6, the head-mountable device 100 can include a controller 602 (e.g., control circuity) with one or more processing units that include or are configured to access a memory 604 having instructions stored thereon. The instructions or computer programs may be configured to perform one or more of the operations or functions described with respect to the head-mountable device 100. The controller 602 can be implemented as any electronic device capable of processing, receiving, or transmitting data or instructions.

For example, the controller 602 may include one or more of: a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitably configured computing element or elements.

The memory 604 can store electronic data that can be used by the head-mountable device 100. For example, the memory 604 can store electrical data or content such as, for example, audio and video files, documents and applications, device settings and user preferences, timing and control signals or data for the various modules, data structures or databases, and so on. The memory 604 can be configured as any type of memory. By way of example only, the memory 604 can be implemented as random access memory, read-only memory, Flash memory, removable memory, or other types of storage elements, or combinations of such devices.

The head-mountable device 100 can further include a display unit 606 for displaying visual information for a user. The display unit 606 can provide visual (e.g., image or video) output. The display unit 606 can be or include an opaque, transparent, and/or translucent display. The display unit 606 may have a transparent or translucent medium through which light representative of images is directed to a user's eyes. The display unit 606 may utilize digital light projection, OLEDs, LEDs, uLEDs, liquid crystal on silicon, laser scanning light source, or any combination of these technologies. The medium may be an optical waveguide, a hologram medium, an optical combiner, an optical reflector, or any combination thereof. In one embodiment, the transparent or translucent display may be configured to become opaque selectively. Projection-based systems may employ retinal projection technology that projects graphical images onto a person's retina. Projection systems also may be configured to project virtual objects into the physical environment, for example, as a hologram or on a physical surface. The head-mountable device 100 can include an optical subassembly configured to help optically adjust and correctly project the image based content being displayed by the display unit 606 for close up viewing. The optical subassembly can include one or more lenses, mirrors, or other optical devices.

The head-mountable device 100 can include an input/output component 610, which can include any suitable component for connecting head-mountable device 100 to other devices. Suitable components can include, for example, audio/video jacks, data connectors, or any additional or alternative input/output components. The input/output component 610 can include buttons, keys, or another feature that can act as a keyboard for operation by the user. Input/output component 610 may include a microphone. The microphone may be operably connected to the controller 602 for detection of sound levels and communication of detections for further processing, as described further herein. Input/output component 610 also may include speakers. The speakers can be operably connected to the controller 602 for control of speaker output, including sound levels, as described further herein.

The head-mountable device 100 can include one or more other sensors 612. Such sensors can be configured to sense substantially any type of characteristic such as, but not limited to, images, pressure, light, touch, force, temperature, position, motion, and so on. For example, the sensor can be a photodetector, a temperature sensor, a light or optical sensor, an atmospheric pressure sensor, a humidity sensor, a magnet, a gyroscope, an accelerometer, a chemical sensor, an ozone sensor, a particulate count sensor, and so on. By further example, the sensor can be a bio-sensor for tracking biometric characteristics, such as health and activity metrics. Other user sensors can perform facial feature detection, facial movement detection, facial recognition, eye tracking, user mood detection, user emotion detection, voice detection, etc. Sensors 612 can include image sensors 105-130 and IMU 160.

The head-mountable device 100 can include communications circuitry 614 for communicating with one or more servers or other devices using any suitable communications protocol. For example, communications circuitry 614 can support Wi-Fi (e.g., a 802.11 protocol), Ethernet, Bluetooth, high frequency systems (e.g., 900 MHz, 2.4 GHz, and 5.6 GHz communication systems), infrared, TCP/IP (e.g., any of the protocols used in each of the TCP/IP layers), HTTP, BitTorrent, FTP, RTP, RTSP, SSH, any other communications protocol, or any combination thereof. Communications circuitry 614 can also include an antenna for transmitting and receiving electromagnetic signals.

The head-mountable device 100 can include a battery 616, which can charge and/or power components of the head-mountable device 100. The battery can also charge and/or power components connected to the head-mountable device 100.

While various embodiments and aspects of the present disclosure are illustrated with respect to a head-mountable device, it will be appreciated that the subject technology can encompass and be applied to other electronic devices. Such an electronic device can be or include a desktop computing device, a laptop-computing device, a display, a television, a portable device, a phone, a tablet computing device, a mobile computing device, a wearable device, a watch, and/or a digital media player.

Implementations within the scope of the present disclosure can be partially or entirely realized using a tangible computer-readable storage medium (or multiple tangible computer-readable storage media of one or more types) encoding one or more instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations are performed by one or more integrated circuits, such as ASICs or FPGAs. In one or more implementations, such integrated circuits execute instructions that are stored on the circuit itself.

According to aspects of the subject technology, a head-mountable device is provided that includes a first image sensor oriented to capture luminance of an area of skin around a user's eye when wearing the head-mountable device and a second image sensor oriented to capture luminance of an area of skin around the user's nose when wearing the head-mountable device. The device further includes a processor configured to determine a pulse signal based on changes in luminance captured over time using at least one of the first image sensor or the second image sensor.

The processor may be further configured to determine the pulse signal based on an average of the changes in luminance captured by the first image sensor and the changes in luminance captured by the second image sensor. The processor may be further configured to filter the changes in luminance captured by the first or second image sensors over time in a first frequency band to obtain the pulse signal. The first image sensor may be an infrared image sensor. The head-mountable device may further include an infrared illuminator oriented to illuminate the area of skin around the user's eye. The head-mountable device may further include a light seal configured to block the area of skin around the user's eye from external light sources.

The head-mountable device may further include a third image sensor oriented to capture images of a portion of the user's chest when wearing the head-mountable device. The second image sensor may be configured to capture images of a user's nostrils when wearing the head-mountable device. The processor may be configured to determine a respiratory signal based on detected motion of the user's nostrils over time using the images captured by the second image sensor or based on detected motion of the user's chest using the images captured by the third image sensor.

a third image sensor oriented to capture luminance of a portion of the user's chest when wearing the head-mountable device. The processor may be further configured to determine a respiratory signal based on changes in luminance captured over time using at least one of the second image sensor or the third image sensor. The processor may be further configured to determine the respiratory signal based on an average of the changes in luminance captured by the second image sensor and the changes in luminance captured by the third image sensor.

The head-mountable device may further include an inertial measurement unit configured to detect motion of the head-mountable device. The processor may be further configured to determine the respiratory signal based on motion of the head-mountable device detected by the inertial measurement unit and the changes in luminance captured by at least one of the second image sensor or the third image sensor. The processor may be further configured to filter the changes in luminance captured by at least one of the second image sensor or the third image sensor over time in a second frequency band to obtain the respiratory signal.

The second image sensor and the third image sensor may be a single image sensor. The second and third image sensors may be infrared image sensors. The head-mountable device may further include an infrared illuminator oriented to illuminate the area of skin around the user's nose and the portion of the user's chest. The head-mountable device may further include a display unit, where the processor may be further configured to provide the pulse signal or the respiratory signal for display to the user on the display unit.

According to aspects of the subject technology, a head-mountable device is provided that includes a first infrared image sensor oriented to capture luminance of an area of skin around a user's eye when wearing the head-mountable device, a second infrared image sensor oriented to capture luminance of an area of skin around the user's nose when wearing the head-mountable device, a third infrared image sensor oriented to capture luminance of a portion of the user's chest when wearing the head-mountable device, and an inertial measurement unit configured to detect motion of the head-mountable device. A processor is configured to determine a pulse signal based on changes in luminance captured over time using at least one of the first infrared image sensor or the second infrared image sensor and a respiratory signal based on changes in luminance captured over time using at least one of the second infrared image sensor or the third infrared image sensor and on motion of the head-mountable device detected by the inertial measurement unit.

The processor may be further configured to determine the pulse signal based on an average of the changes in luminance captured by the first infrared image sensor and the changes in luminance captured by the second infrared image sensor. The processor may be further configured to determine the respiratory signal based on an average of the changes in luminance captured by the second infrared image sensor and the changes in luminance captured by the third infrared image sensor. The processor may be further configured to filter the changes in luminance captured by at least one of the first infrared image sensor or the second infrared image sensor over time in a first frequency band to obtain the pulse signal. The processor may be further configured to filter the changes in luminance captured by at least one of the second infrared image sensor or the third infrared image sensor over time in a second frequency band to obtain the respiratory signal.

According to aspects of the subject technology, a method is provided that includes capturing periodically a first plurality of images of an area of skin around a user's eye with a first image sensor of a head-mountable device worn by the user; determining a luminance value for each of the first plurality of images to generate a first luminance signal; filtering the first luminance signal by a first frequency band to determine a heart rate; and providing the heart rate for display.

The method may further include capturing periodically a second plurality of images of an area of skin around the user's nose with a second image sensor of a head-mountable device worn by the user; determining a luminance value for each of the second plurality of images to generate a second luminance signal; and averaging the first luminance signal and the second luminance signal to generate a first average luminance signal. The first average luminance signal is filtered by the first frequency band to determine the heart rate.

The method may further include capturing periodically a third plurality of images of a portion of the user's chest with a third image sensor of the head-mountable device worn by the user; determining a luminance value for each of the third plurality of images to generate a third luminance signal; filtering the third luminance signal by a second frequency band to determine a respiratory rate; and providing the respiratory rate for display on the display of the head-mountable device worn by the user. The method may further include averaging the second luminance signal and the third luminance signal to generate a second average luminance signal, where the second average luminance signal is filtered by the second frequency band to determine the respiratory rate. The method may further include capturing periodically motion of the head-mountable device using an inertial measurement unit; and combining the captured motion with the third luminance signal, where the combined capture motion and third luminance signal is filtered by the second frequency band to determine the respiratory rate.

As described herein, aspects of the present technology can include the gathering and use of data. The present disclosure contemplates that in some instances, gathered data can include personal information or other data that uniquely identifies or can be used to locate or contact a specific person. The present disclosure contemplates that the entities responsible for the collection, analysis, disclosure, transfer, storage, or other use of such personal information or other data will comply with well-established privacy practices and/or privacy policies. The present disclosure also contemplates embodiments in which users can selectively block the use of or access to personal information or other data (e.g., managed to minimize risks of unintentional or unauthorized access or use).

A reference to an element in the singular is not intended to mean one and only one unless specifically so stated, but rather one or more. For example, “a” module may refer to one or more modules. An element proceeded by “a,” “an,” “the,” or “said” does not, without further constraints, preclude the existence of additional same elements.

Headings and subheadings, if any, are used for convenience only and do not limit the invention. The word exemplary is used to mean serving as an example or illustration. To the extent that the term include, have, or the like is used, such term is intended to be inclusive in a manner similar to the term comprise as comprise is interpreted when employed as a transitional word in a claim. Relational terms such as first and second and the like may be used to distinguish one entity or action from another without necessarily requiring or implying any actual such relationship or order between such entities or actions.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A phrase “at least one of” preceding a series of items, with the terms “and” or “or” to separate any of the items, modifies the list as a whole, rather than each member of the list. The phrase “at least one of” does not require selection of at least one item; rather, the phrase allows a meaning that includes at least one of any one of the items, and/or at least one of any combination of the items, and/or at least one of each of the items. By way of example, each of the phrases “at least one of A, B, and C” or “at least one of A, B, or C” refers to only A, only B, or only C; any combination of A, B, and C; and/or at least one of each of A, B, and C.

It is understood that the specific order or hierarchy of steps, operations, or processes disclosed is an illustration of exemplary approaches. Unless explicitly stated otherwise, it is understood that the specific order or hierarchy of steps, operations, or processes may be performed in different order. Some of the steps, operations, or processes may be performed simultaneously. The accompanying method claims, if any, present elements of the various steps, operations or processes in a sample order, and are not meant to be limited to the specific order or hierarchy presented.

These may be performed in serial, linearly, in parallel or in different order. It should be understood that the described instructions, operations, and systems can generally be integrated together in a single software/hardware product or packaged into multiple software/hardware products.

In one aspect, a term coupled or the like may refer to being directly coupled. In another aspect, a term coupled or the like may refer to being indirectly coupled.

Terms such as top, bottom, front, rear, side, horizontal, vertical, and the like refer to an arbitrary frame of reference, rather than to the ordinary gravitational frame of reference. Thus, such a term may extend upwardly, downwardly, diagonally, or horizontally in a gravitational frame of reference.

The disclosure is provided to enable any person skilled in the art to practice the various aspects described herein. In some instances, well-known structures and components are shown in block diagram form in order to avoid obscuring the concepts of the subject technology. The disclosure provides various examples of the subject technology, and the subject technology is not limited to these examples. Various modifications to these aspects will be readily apparent to those skilled in the art, and the principles described herein may be applied to other aspects.

All structural and functional equivalents to the elements of the various aspects described throughout the disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for”.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirements of the applicable patent law, nor should they be interpreted in such a way.

1. A head-mountable device, comprising:
a first image sensor oriented to capture a first luminance of a first area of skin around an eye;
a second image sensor oriented to capture a second luminance of a second area of skin around a nose; and
a processor configured to determine a pulse signal based on changes in at least one of the first luminance or the second luminance captured over time using at least one of the first image sensor or the second image sensor.

2. The head-mountable device of claim 1, wherein the processor is further configured to determine the pulse signal based on an average of the changes in the first luminance captured by the first image sensor and the changes in the second luminance captured by the second image sensor.

3. The head-mountable device of claim 1, wherein the processor is further configured to filter the changes in at least one of the first luminance or the second luminance captured by the first or second image sensors over time in a first frequency band to obtain the pulse signal.

4. The head-mountable device of claim 1, wherein the first image sensor is an infrared image sensor.

5. The head-mountable device of claim 4, further comprising an infrared illuminator oriented to illuminate the first area of skin around the eye.

6. The head-mountable device of claim 1, further comprising a light seal configured to block the first area of skin around the eye from external light sources.

7. The head-mountable device of claim 1, further comprising:
a third image sensor oriented to capture images of a portion of a chest,
wherein the second image sensor is further configured to capture images of nostrils, and
wherein the processor is further configured to determine a respiratory signal based on detected motion of the nostrils over time using the images captured by the second image sensor or based on detected motion of the chest using the images captured by the third image sensor.

8. The head-mountable device of claim 7, further comprising:
an inertial measurement unit configured to detect motion of the head-mountable device,
wherein the processor is further configured to determine the respiratory signal based on the motion of the head-mountable device detected by the inertial measurement unit and the changes in at least one of the first luminance or the second luminance captured by at least one of the second image sensor or the third image sensor.

9. The head-mountable device of claim 7, wherein the processor is further configured to filter the changes in at least one of the first luminance or the second luminance captured by at least one of the second image sensor or the third image sensor over time in a second frequency band to obtain the respiratory signal.

10. The head-mountable device of claim 7, wherein the second image sensor and the third image sensor are a single image sensor.

11. The head-mountable device of claim 7, wherein the second and third image sensors are infrared image sensors.

12. The head-mountable device of claim 11, further comprising an infrared illuminator oriented to illuminate the second area of skin around the nose and the portion of the chest.

13. The head-mountable device of claim 7, further comprising:
a display unit,
wherein the processor is further configured to provide at least one of the pulse signal or the respiratory signal for display on the display unit.

14. A head-mountable device, comprising:
a first infrared image sensor oriented to capture a first luminance of a first area of skin around an eye;
a second infrared image sensor oriented to capture a second luminance of a second area of skin around a nose;
a third infrared image sensor oriented to capture a third luminance of a portion of a chest;
an inertial measurement unit configured to detect motion of the head-mountable device; and
a processor configured to determine (1) a pulse signal based on changes in at least one of the first luminance or the second luminance captured over time using at least one of the first infrared image sensor or the second infrared image sensor and (2) a respiratory signal based on changes in at least one of the second luminance or the third luminance captured over time using at least one of the second infrared image sensor or the third infrared image sensor and on the motion of the head-mountable device detected by the inertial measurement unit.

15. The head-mountable device of claim 14, wherein the processor is further configured to determine the pulse signal based on an average of the changes in the first luminance captured by the first infrared image sensor and the changes in the second luminance captured by the second infrared image sensor.

16. The head-mountable device of claim 14, wherein the processor is further configured to determine the respiratory signal based on an average of the changes in the second luminance captured by the second infrared image sensor and the changes in the third luminance captured by the third infrared image sensor.

17. The head-mountable device of claim 14, wherein the processor is further configured to filter the changes in at least one of the first luminance or the second luminance over time in a first frequency band to obtain the pulse signal.

18. The head-mountable device of claim 14, wherein the processor is further configured to filter the changes in at least one of the second luminance or the third luminance over time in a second frequency band to obtain the respiratory signal.

19. A method, comprising:
capturing periodically a first plurality of images of a first area of skin around an eye with a first image sensor of a head-mountable device;
determining a first luminance value for each of the first plurality of images to generate a first luminance signal;
capturing periodically a second plurality of images of a second area of skin around a nose with a second image sensor of the head-mountable device;
determining a second luminance value for each of the second plurality of images to generate a second luminance signal;
averaging the first luminance signal and the second luminance signal to generate a first average luminance signal;
filtering the first average luminance signal by a first frequency band to determine a heart rate; and
providing the heart rate for display.

20. The method of claim 19, further comprising:
capturing periodically a third plurality of images of a portion of a chest with a third image sensor of the head-mountable device;
determining a third luminance value for each of the third plurality of images to generate a third luminance signal;
filtering the third luminance signal by a second frequency band to determine a respiratory rate; and
providing the respiratory rate for display on a display unit of the head-mountable device.

21-22. (canceled)
